(19) United States
(12) Patent Application Publication
(10) Pub. No.: US 2015/0185129 A1
(43) Pub. Date: Jul. 2, 2015

(54) METHODS AND DEVICES FOR TESTING THE STABILITY OF INTRALUMINAL IMPLANTS

(71) Applicants: Karmi Nicole Robison, Vista, CA (US); Alexander Joseph Asconeguy, Murrieta, CA (US)

(72) Inventors: Karmi Nicole Robison, Vista, CA (US); Alexander Joseph Asconeguy, Murrieta, CA (US)

(21) Appl. No.: 14/457,390

(22) Filed: Aug. 12, 2014

Related U.S. Application Data
(60) Provisional application No. 61/865,577, filed on Aug. 13, 2013.

(51) Int. Cl. G01N 3/02; G01N 3/08

(52) U.S. Cl. CPC G01N 3/02; G01N 3/08; A61F 2250/0096; A61F 2002/488

(57) ABSTRACT

According to some embodiments, a method of testing the stability of an implant configured for placement within a target vessel or other body lumen of a subject includes positioning an implant within a test conduit, the test conduit comprising a wall defining an interior opening, wherein the implant at least partially contacts an interior surface of the wall when positioned within the interior opening of the test conduit, applying a force to at least a portion of the implant, measuring a longitudinal extension of the implant at various increasing levels of force applied to the implant and comparing the longitudinal extension of the implant relative to the force applied to the implant to evaluate a stability of the implant.

### CROSS-REFERENCE TO PRIORITY APPLICATIONS AND RELATED APPLICATIONS

This application claims benefit of priority to U.S. Provisional Patent Application Ser. No. 61/865,577, filed Aug. 13, 2013, incorporated by reference herein. This application is also related to PCT/US14/47366, filed July 21, 2014, U.S. patent application Ser. No. 13/106,343, filed May 12, 2011 and published as U.S. Publication No. 2011/0282343, U.S. patent application Ser. No. 13/457,033, filed Apr. 26, 2012 and published as U.S. Publication No. 2012/0277842, U.S. patent application Ser. No. 13/655,351, filed Oct. 18, 2012 and published as U.S. Publication No. 2013/0109987, U.S. patent application Ser. No. 13/830,040, filed Mar. 14, 2013 and U.S. Provisional Patent Application No. 61/856,598, filed Jul. 19, 2013, are expressly incorporated by reference herein and made a part of the present application.

### BACKGROUND

1. Field

This application relates to methods of testing the stability of intraluminal (e.g., intravascular) implants, as well as various tools, devices and systems related thereto.

2. Description of the Related Art

In some embodiments, it is desirable for intraluminal implants to have a threshold level of stability (e.g., lateral stability). Such implants can include implants comprising a coiled or helically shaped ribbon, stents, other implantable devices and/or the like. Accurate stability testing of implants can help ensure that implants will remain coaxially aligned with a target lumen (e.g., vein, artery, other vessel, airway, etc.) after implantation. Accordingly, various test methods and devices are disclosed herein for evaluating the stability of implants.

### SUMMARY

According to some embodiments, a method of testing the stability (e.g., lateral stability) of an implant configured for placement within a target vessel or other body lumen of a subject comprises positioning an implant within a test conduit, the test conduit comprising a wall defining an interior opening, wherein the implant at least partially contacts an interior surface of the wall when positioned within the interior opening of the test conduit, applying a longitudinal force to at least a portion of the implant using a force applicator and evaluating a resistive force by the implant on the test conduit over a range of increasing longitudinal force applied to the implant by the force applicator. In some embodiments, evaluating a resistive force by the implant on the test conduit over a range of increasing longitudinal force applied to the implant helps determine whether the implant maintains its longitudinal orientation within the test conduit.

According to some embodiments, the force applicator comprises a device that is configured to be selectively moved toward the implant and to engage the test conduit. In one embodiment, the force applicator comprises a curved or rounded shape along a location where the force applicator contacts the test conduit. In other embodiments, the force applicator is configured to apply a force to the implant without physically contacting the test conduit and/or the implant (e.g., via a pneumatic force, other pressure-inducing device or method, etc.). In some embodiments, the force applied to the at least a portion of the implant comprises both a radial force component and a longitudinal force component. In some embodiments, the curvature and/or other shape or feature of the force applicator helps determine the distribution of force in the radial and axial directions.

According to some embodiments, evaluating a resistive force by the implant on the test conduit over a range of increasing longitudinal force applied to the implant comprises plotting or otherwise comparing the longitudinal force applied to the implant against a parameter associated with lateral stability of the implant in a graph. In some embodiments, the parameter associated with lateral stability of the implant comprises a resistive force by the implant radially on an interior of the test conduit.

According to some embodiments, the method further comprises repeating a test procedure for the same implant to evaluate the reproducibility of the comparison of the resistive force by the implant radially on the interior of the test conduit relative to the longitudinal force applied to the implant. In some embodiments, the method further comprises calculating an average coefficient of variation between the various different test procedures. In some embodiments, the method additionally comprises determining whether the average coefficient of variation is less or greater than about 20 (e.g., 0-20, 0-10, 0-5, etc.). In some embodiments, the method further comprises calculating a coefficient of determination between the various different test procedures. In some embodiments, the method additionally comprises determining whether the coefficient of determination is less or greater than about 0.7 (e.g., 0.7-0.75, 0.75-0.8, 0.8-0.85, 0.85-0.9, greater than 0.9, values between the foregoing ranges, etc.), 0.4 (e.g., 0.4-0.45, 0.45-0.5, values within the foregoing ranges, etc.), above 0.5 (e.g., (e.g., 0.5-0.55, 0.55-0.6, values within the foregoing ranges, etc.), above 0.6 (e.g., 0.6-0.65, 0.65-0.7, values within the foregoing ranges, etc.) and/or the like.

According to some embodiments, evaluating a resistive force by the implant on the test conduit over a range of increasing longitudinal force applied to the implant comprises determining whether a resistive force by the implant on the test conduit in the radial direction decreases as the longitudinal force applied to the implant increases.

According to some embodiments, the implant comprises a ribbon that includes, at least in part, a helical or wound shape. In some embodiments, the implant comprises a wire or other structure. In some embodiments, the implant comprises a stent or any other intraluminal device configured to at least partially contact an adjacent portion of a body lumen (e.g., vein, artery, other blood vessel, other vessel, airway, etc.) when implanted in a subject. In some embodiments, applying a force to at least a portion of the implant comprises applying a force at, along or near one end of the implant. In some embodiments, a diameter or other cross-section dimension of the implant being tested is between 1 mm and 50 mm (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 30-35, 35-40, 40-45, 45-50 mm, diameters between the foregoing values or ranges, etc.). In some embodiments, the diameter or other cross-section dimension of the implant being tested is between 2 mm and 20 mm.

According to some embodiments, a stability being evaluated comprises, at least in part, a lateral stability of the implant. In some embodiments, the method further comprises adjusting at least one parameter of the implant being tested to improve the stability of the implant. In some embodiments, adjusting at least one parameter of the implant comprises increasing the aspect ratio of the implant, increasing a width of a winding or element of the implant, increasing a length of the implant and/or the like.

According to some embodiments, a method of improving a lateral stability of an intraluminal implant comprises testing a stability of an implant according to any of the testing embodiments disclosed herein, and adjusting at least one parameter of the implant being tested to improve the stability of the implant. In some embodiments, adjusting at least one parameter of the implant comprises increasing the aspect ratio of the implant, increasing a width of a winding or element of the implant, increasing a length of the implant and/or the like.

According to some embodiments, a device for testing a lateral stability of an implant comprises a test conduit (e.g., tube, other cylindrical member, etc.) comprising a wall defining an interior opening configured to receive an implant, wherein the implant at least partially contacts an interior surface of the wall when positioned within the interior opening of the test conduit, and a force applicator configured to contact at least a portion of the test conduit and configured to apply a force to at least a portion of the implant.

In some embodiments, the force application is configured to impart, at least in part, a lateral force component to the implant. In one embodiment, a parameter associated with the lateral stability of the implant within the test conduit is configured to be evaluated at various increasing levels of force applied to the implant.

According to some embodiments, the force applicator comprises a curved, rounded shape or irregular shape along a location where the force applicator contacts the test conduit. In some embodiments, the force applicator is configured to be selectively moved relative to the test conduit. In some embodiments, the force applicator is configured to contact the implant and/or the test conduit. In other embodiments, the force application is configured to apply the desired force to the test conduit and the implant without physically contacting the test conduit and/or the implant being tested (e.g., pneumatically, via air pressure, other non- contact pressure or force inducing device or method, etc.). In some embodiments, the force application is configured to be moved using at least one of a pneumatic, hydraulic and mechanical device. In some embodiments, the device further comprises a platform configured to receive the test conduit during a testing procedure. In some embodiments, the parameter associated with the lateral stability of the implant within the test conduit comprises a resistive force by the implant radially on an interior surface of the test conduit.

The methods summarized above and set forth in further detail below describe certain actions taken by a practitioner; however, it should be understood that they can also include the instruction of those actions by another party. Thus, actions such as “applying a force” include “instructing the application of a force.”

### BRIEF DESCRIPTION OF THE DRAWINGS

These and other features, aspects and advantages of the present application are described with reference to drawings of certain embodiments, which are intended to illustrate, but not to limit, the various inventions disclosed herein. It is to be understood that the attached drawings are for the purpose of illustrating concepts and embodiments of the present application and may not be to scale.

FIG. 1 schematically illustrates the application of forces on an intraluminal implant positioned within a target body lumen according to one embodiment;

FIG. 2 illustrates one embodiment of a testing device configured to evaluate the stability of an intraluminal implant;

FIG. 3 is a detailed view of the testing device illustrated in FIG. 2;

FIGS. 4A and 4B illustrate embodiments of load curves generated using data obtained from the stability testing methods and devices disclosed herein; and

FIG. 5 illustrates a flowchart that schematically illustrates various embodiments for testing the stability of intraluminal implants.

### DETAILED DESCRIPTION

The discussion and the figures illustrated and referenced herein describe various embodiments of evaluating the stability (e.g., lateral stability) of an intraluminal (e.g., intravascular) implant, as well as various tools, systems and methods related thereto. According to some embodiments, the implant being tested comprises a ribbon that has a generally helical or similarly wound shape. Such implants can be configured for placement within a target body lumen (e.g., a pulmonary vein, a renal artery, an airway, etc.) and may be particularly well suited to treat atrial fibrillation, other cardiac arrhythmias, renal-induced hypertension, asthma, COPD, gastroenterological diseases and conditions (e.g., GERD), urinary tract conditions and/or the like. However, the implants being tested according to the various embodiments disclosed herein can include a different structure and/or may be configured to treat any other type of disease or condition. For example, the methods, devices and systems disclosed herein can be used to evaluate the stability (e.g., lateral stability) of a stent or other device that is configured to be implanted within a body lumen of a subject.

According to some embodiments, methods and devices for measuring the stability (e.g., lateral stability) of an intraluminal implant are disclosed herein. By measuring the stability of vascular implants, a determination can be made for predicting how well an implant will remain within a target body lumen (e.g., vein, artery, other vessel, airway, etc.) of a subject. Specifically, the test methods and devices disclosed herein allow for an accurate assessment of how well an implant will remain co-axially deployed within a target lumen after implantation.

The embodiments disclosed herein provide certain advantages and benefits over prior procedures. For example, in some embodiments, the testing methods and devices described herein permit for a qualitative and/or a quantitative evaluation of stability. Accordingly, the relative stability (e.g., lateral stability) of various implant designs can be more accurately determined and evaluated (e.g., at an early stage of the design process). This can facilitate the initial design efforts of luminal implants (e.g., to eliminate certain embodiments based on stability). Further, the need for relatively tedious, unreliable, time consuming and costly trial and error testing methods can be advantageously eliminated.

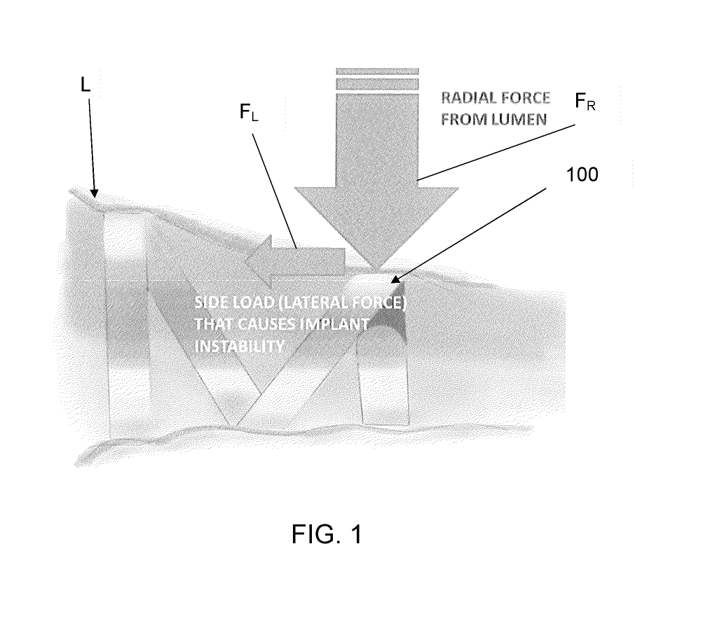

FIG. 1 schematically illustrates one embodiment of an implant 100 positioned within a target body lumen L (e.g., vein, artery, other blood vessel, airway, urinary tract lumen, gastroenterological tract lumen, other body canals or cavities, etc.). As shown, the implant 100 can comprise a ribbon that is wound about a central axis. However, as discussed in greater detail herein, the implant 100 being tested can include any other shape, configuration or design. For instance, the implant can include a stent or any other device that is configured to at least partially contact the interior surface of the lumen L within which is placed. Once deployed within the lumen L, the at least a portion of the implant 100 (e.g., the ribbon) can radially expand or otherwise deploy so as to contact the interior surface of the lumen. As a result of such a radial deployment, in some embodiments, a resistive force is imparted on the implant 100 by the lumen L.

With continued reference to FIG. 1, the resistive force imparted on the implant 100 can include both a radial force component F_{R} and a lateral (or axial or longitudinal) force component F_{L}. As shown, the lateral force component F_{R} is in a direction parallel or generally parallel with the longitudinal axis of the implant 100. In some embodiments, the implant 100 may be susceptible to turning over or otherwise moving out of coaxial alignment with the lumen due to, at least in part, the lateral force component F_{L}. Thus, in some embodiments, lateral instability of an implant can be caused by the lateral forces exerted on the implant by the adjacent portions of the target body lumen. In some embodiments, a higher level of lateral stability results in a greater resistance of the implant against overturning or otherwise losing its coaxial alignment with the lumen after implantation. Accordingly, in some embodiments, a testing device or system can be used to strategically apply forces to the implant (e.g., over a varying range of forces) so as to simulate the loads that may be applied to the implant after implantation in a body lumen. Therefore, as discussed in greater detail herein, the relative stability of an implant can be evaluated.

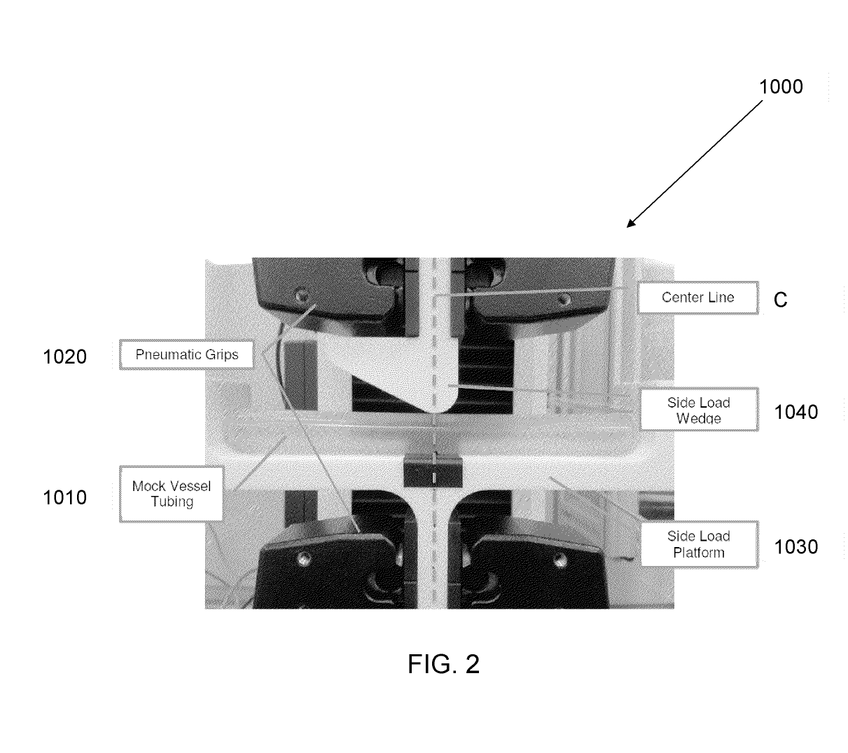

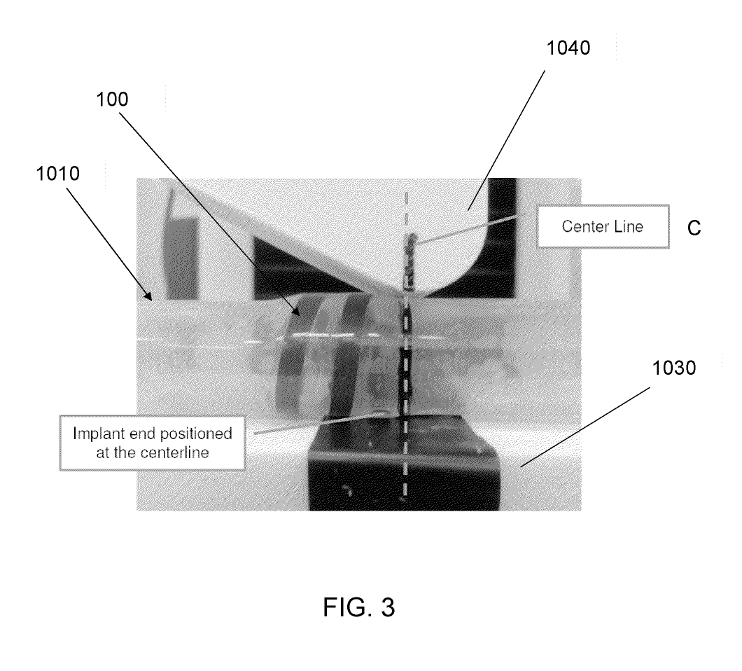

One embodiment of a device 1000 configured to evaluate the stability of intraluminal implant is illustrated in FIGS. 2 and 3. As shown, the testing device 1000 can include a test conduit 1010 (e.g., mock vessel tubing, other tube or sleeve, etc.) that is configured to receive an implant being tested. The test conduit 1010 can comprise a generally cylindrical shape with a circular cross-section shape. However, the test conduit 1010 can include any other shape, as desired or required. For example, in some embodiments, the test conduit 1010 comprises an oval or irregular cross-sectional shape. Thus, the test conduit 1010 can be designed to simulate a native body lumen (e.g., vein, artery, other vessel, airway, etc.) of a subject in which the implant is configured to be implanted. In some embodiments, the interior surface of the test conduit 1010 can include undulations, discontinuities, other features or imperfections (e.g., bumps, rings, recesses, etc.) and/or the like so as to more accurately reflect a native body lumen. Likewise, the diameter or other cross-sectional dimension of the test conduit 1010 can be uniform along the length of the conduit or it can vary, as desired or required.

In some embodiments, the test conduit comprises one or more flexible, semi-rigid and/or rigid materials, such as, for example, plastic, rubber, other natural or synthetic materials. As depicted in FIGS. 2 and 3, the test conduit can be at least partially transparent or translucent (e.g., because of the materials used, one or more viewing windows or features, etc.) to advantageously permit a user of the testing device to visually identify an implant positioned within an interior of the conduit 1010.

With continued reference to FIGS. 2 and 3, in some embodiments, the test conduit 1010 can be positioned between a force applicator 1040 (e.g., a side load wedge, another movable contacting surface, component or feature, etc.) and a platform 1030 (e.g., a side load platform). In some embodiments, the force applicator 1040 is positioned above the platform 1030 and is configured to be movable relative to the platform. However, in other embodiments, the orientation of the force applicator and platform can be different than illustrated herein (e.g., reversed, positioned horizontally, diagonally, etc.). Further, is some embodiments, the platform 1030 is movable relative to the force applicator 1040, either in addition to or in lieu of the force applicator being movable.

In some embodiments, the platform 1030 of the device 1000 can comprise a generally flat upper surface on which the test conduit 1010 is configured to be placed. In other embodiments, the upper surface of the platform 1030 can be at least partially circular or curved (e.g., so as to match or generally match the shape of the test conduit) or can comprise any other shape or configuration, as desired or required. The test conduit 1010 can be removably or fixedly positioned on the platform. In some embodiments, one or more fasteners or other attachment devices (e.g., straps, bands, coupling devices, tabs, screws, rivets, adhesives, etc.) can be used to secure (e.g., removably or fixedly secure) the test conduit to the platform 1030.

As illustrated in FIGS. 2 and 3, the lower surface of the force applicator 1040 can include a smooth, rounded shape. This can help to exert a radial and a lateral (e.g., longitudinal or axial) load on the implant and/or to more evenly distribute the forces applied to an implant being tested (e.g., reduce or eliminate isolated or localized loads on the implant at or near the point of contact). The terms lateral, axial and longitudinal force or load are used interchangeably herein. However, in other embodiments, the shape of the force applicator can vary, as desired or required. For example, the surface of the force applicator 1040 that is configured to contact the test conduit 1010 (e.g., the lower surface of the force applicator) can include a flat shape (e.g., such that the lower surface is parallel or generally parallel with the centerline of the test conduit) or any other shape (e.g., non-flat, non-smooth, fluted, irregular, recessed, bumpy, pointed, undulating, etc.).

With continued reference to FIGS. 2 and 3, the force applicator 1040 can include a centerline C that generally coincides or aligns with the point of contact with the adjacent outer surface of the test conduit 1010 when the force applicator is moved relative to the test conduit. Thus, in some embodiments, the centerline C of the force applicator is also the centerline of the load or force that is applied to the test conduit 1010 and an implant positioned within the test conduit. Once an implant has been positioned within the test conduit, the device 1000 can be selectively moved to initiate a testing procedure. For example, in some embodiments, the force applicator 1040 can be lowered relative to the test conduit 1010 so as to apply a force or a range of forces on the test conduit and the implant secured therein. As noted above, in other embodiments, the platform can be moved to urge the test conduit 1010 toward the force applicator, either in lieu of or in addition to moving the force applicator 1040.

The force applicator 1040 and/or the platform 1030 of the testing device 1000 can be moved using one or more mechanical, hydraulic, pneumatic or other devices. For example, in some embodiments, as illustrated in FIG. 2, one or more pneumatic grips 1020 can be used to move the various components of the testing device 1000 in a desired manner (e.g., to move the force application toward the test conduit and the implant positioned therein). In other embodiments, one or more motors (e.g., stepper motors, gear drives, etc.) and/or any other mechanical, electromechanical and/or other movable device can be used. Regardless of the exact manner in which the components of the device 1000 are moved relative to each other, in some embodiments, the force applicator 1040 is configured to be moved relative to the test conduit 1010 in a predictable and uniform manner (e.g., continuously or intermittently) so as to apply a steadily increasing force or load on the test conduit 1010 and the implant positioned therein. In other embodiments, however, the force applicator can be configured to move in a non-uniform manner (e.g., with varying acceleration or speed), as desired or required.

As illustrated in FIG. 3, the implant 100 being tested can be positioned within the test conduit 1010. In some embodiments, test conduit 1010 is sized so that the implant, when radially deployed, can contact the interior surface of the test conduit. Accordingly, the device 1000 can include test conduits 1010 of varying size, shape and/or configuration in order to accommodate the various implants being tested. For example, in some embodiments, the diameter of the deployed implant being tested can vary between about 1 mm and about 50 mm (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 30-35, 35-40, 40-45, 45-50 mm, diameters between the foregoing values or ranges, etc.). In other embodiments, the diameter of the implant being tested using the device 1000 can be less than about 1 mm or greater than about 50 mm. Therefore, the test conduit 1010 can comprise an inner diameter or other cross- section dimension that also varies between about 1 mm and 50 mm (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 30-35, 35-40, 40-45, 45-50 mm, diameters between the foregoing values or ranges, etc.). Likewise, the inner diameter or other cross-sectional dimension of the test conduit 1010 can be less than about 1 mm or greater than about 50 mm, as desired or required.

With continued reference to FIG. 3, in some embodiments, the test conduit 1010 is strategically positioned between the upper force applicator 1040 and the lower platform 1030 so that at least a portion of the implant 100 situated within test conduit is generally aligned with the centerline C of the force applicator. For example, as illustrated in FIG. 3, in some embodiments, the centerline C of the force applicator 1040 is aligned or generally aligned with one end of the implant 100 (e.g., the ribbon of the implant 100 that forms one end of the implant). However, in other embodiments, the centerline C of the force applicator can be aligned with any other portion of the implant being tested (e.g., the middle of the implant, another location between the ends of the implant, etc.). In some embodiments, the various implants being tested are aligned in a similar manner relative to the force applicator so as to evaluate the relative stability of the implants.

In some embodiments, once the test conduit 1010 (and thus, the implant 100) has been properly aligned or otherwise oriented relative to the force applicator 1040, the load application procedure can be initiated. The force applicator 1040 can be configured to be lowered or otherwise moved (e.g., either continuously or intermittently, in a step-wise fashion, etc.) relative to the test conduit 1010. Eventually, in some embodiments, the force applicator 1040 contacts the outer surface of the test conduit 1010 and applies a force or load to the test conduit and the implant 100 positioned therein. In some embodiments, the curved lower surface of the force applicator 1040 is specifically shaped so that the radial force component F_{R} and the lateral force component F_{L} of the force applied to the implant can be computed. Thus, the shape of the portion of the force applicator that is configured to contact the test conduit 1010 can be selected to vary the relative distribution of the radial and lateral components of the force applied to the implant. For example, in some embodiments, the percentage of the lateral or longitudinal force component F_{L} relative to the total force applied to the implant 100 can be about 20% to 100% (e.g., 20-25%, 25-30%, 30-35%, 35-40%, 40-45%, 45-50%, 50-55%, 55-60%, 60-65%, 65-70%, 70-75%, 75-80%, 80-85%, 85-90%, 90-95%, 95-100%, values between the foregoing, etc.). In addition, the total force or load applied to the implant 100 by the force applicator 1040 during a testing procedure can vary between 0 and 90 N (e.g., 0-1, 1-2, 2-3, 3-4, 4-5, 5-6, 6-7, 7-8, 8-9, 9-10, 10-12, 12-14, 14-16, 16-18, 18-20, 20-22, 22-24, 24-26, 26-28, 28-30, 30-35, 35-40, 40-45, 45-50, 50-55, 55-60, 60-65, 65-70, 70-75, 75-80, 80-85, 85-90 N, forces between the foregoing ranges, etc.). Thus, in some embodiments, the lateral or longitudinal force component applied to the implant 100 by the force applicator 1040 during a testing procedure can vary between 0 and 90 N (e.g., 0-1, 1-2, 2-3, 3-4, 4-5, 5-6, 6-7, 7-8, 8-9, 9-10, 10-12, 12-14, 14-16, 16-18, 18-20, 20-22, 22-24, 24-26, 26-28, 28-30, 30-35, 35-40, 40-45, 45-50, 50-55, 55-60, 60-65, 65-70, 70-75, 75-80, 80-85, 85-90 N, forces between the foregoing ranges, etc.). The shape of the force applicator's lower surface can be modified in order to alter the distribution of force between.

According to some embodiments, the amount of force (e.g., lateral or longitudinal force) that is applied to the implant 100 during a testing procedure can be measured against the extension or travel distance of the force applicator 1040 (e.g., the distance by which the force application is moved toward the implant being tested). According to some embodiments, the travel distance of the force applicator correlates to a resistive force by the implant on the test conduit 1010 in the radial direction. Thus, in some embodiments, maintaining a resistive radial force by the implant on the adjacent portions of the test conduit suggests that the implant retains its longitudinal position within the test conduit, and thus, is laterally stable therein. The force applied by the force applicator to the implant can be conducted by physical contact or otherwise (e.g., air pressure).

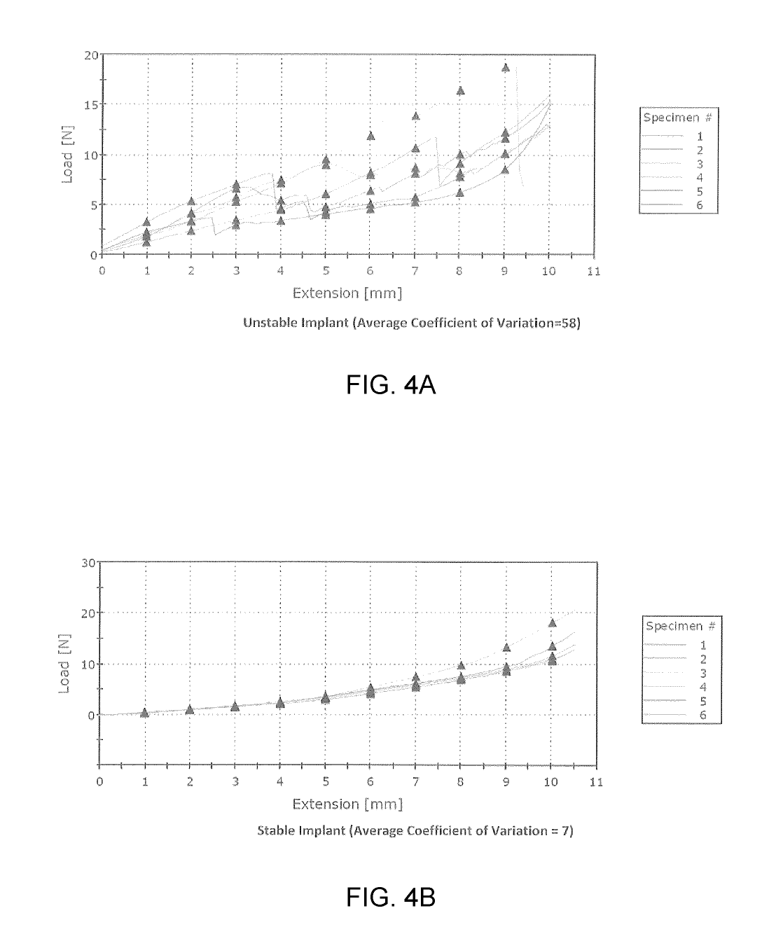

In some embodiments, a comparison of lateral force applied to the implant being tested versus a parameter of resulting stability of the implant (e.g., a resistive force by the implant on the test conduit) can be used to create corresponding curves, such as those illustrated in FIGS. 4A and 4B. For example, FIG. 4B illustrates the load curve for a relatively stable implant, since the curve has no decreases or other sudden changes in resistive force by the implant on the adjacent surface of the test conduit during the compressive loading of the implant being tested (e.g., as the force applicator is moved toward and against the implant, and thus, the lateral load on the implant is increased). As noted herein, one or more other stability parameters can be used in a testing evaluation, either in lieu of or in addition to resistive radial force by the implant on the test conduit. In some embodiments, decreases or other abrupt changes in the resistive force of the implant on the test conduit relative to movement of the force applicator toward the implant (e.g., or other method or mechanism of increasing the lateral load on the implant being tested) during a testing procedure are associated with the implant at least partially overturning or otherwise changing its longitudinal axis relative to the lumen within which it is positioned (e.g., the test conduit 1010 for purposes of the test method).

In addition, according to some embodiments, a relatively unstable implant can be associated with unpredictability or non-repeatability between similar tests of the same implant. For example, the test results for such an implant are illustrated in FIG. 4A. As shown, the load curves vary significantly between the different test runs, despite the fact that the same implant was tested. In the illustrated embodiment, for example, the average coefficient of variation between the various test procedures was approximately 58.

In contrast, FIG. 4B illustrates load curves associated with a relatively stable implant tested in accordance with the embodiments disclosed herein. In the depicted embodiment, the load curves are generally smooth (e.g., there are no decreases or other abrupt variations in the resistive or other stability parameter), suggesting that the implant is not susceptible to overturning, torqueing or otherwise undesirably moving within the target lumen, at least within the tested range. Therefore, one threshold or prerequisite of stability for an implant being tested is that there are no decreases or other abrupt changes in load or force by the implant on the adjacent test conduit as the force applicator is moved toward the implant during the execution of a test procedure.

In addition, in the embodiment of FIG. 4B, the load curves are generally predictable between the sequential test runs. For example, in the illustrated embodiment, the average coefficient of variation of the different curve iterations was approximately 7. In some embodiments, a target average coefficient of variation in load curves associated with a series of test different runs of the same implant below about 20 (e.g., 0-20, 0-10, 0-5, etc.) provides assurance that the implant has satisfied a minimum stability threshold. In some embodiments, the coefficient of determination for the sequential sample test run's data set can also be used as a measure of implant stability performance. By way of example, the coefficient of determination of the dataset illustrated in FIG. 4B is approximately 0.8 to 0.9 (e.g., 0.88). In some embodiments, a coefficient of determination of the sample data set consisting of a series of test runs of the same implant greater than 0.7 (e.g., 0.7-0.75, 0.75-0.8, 0.8-0.85, 0.85-0.9, greater than 0.9, values between the foregoing ranges, etc.) can provide assurance that the implant has satisfied a desired or target stability threshold. In other embodiments, the target coefficient of determination is above 0.4 (e.g., 0.4-0.45, 0.45-0.5, values within the foregoing ranges, etc.), above 0.5 (e.g., (e.g., 0.5-0.55, 0.55-0.6, values within the foregoing ranges, etc.), above 0.6 (e.g., 0.6-0.65, 0.65-0.7, values within the foregoing ranges, etc.) and/or the like.

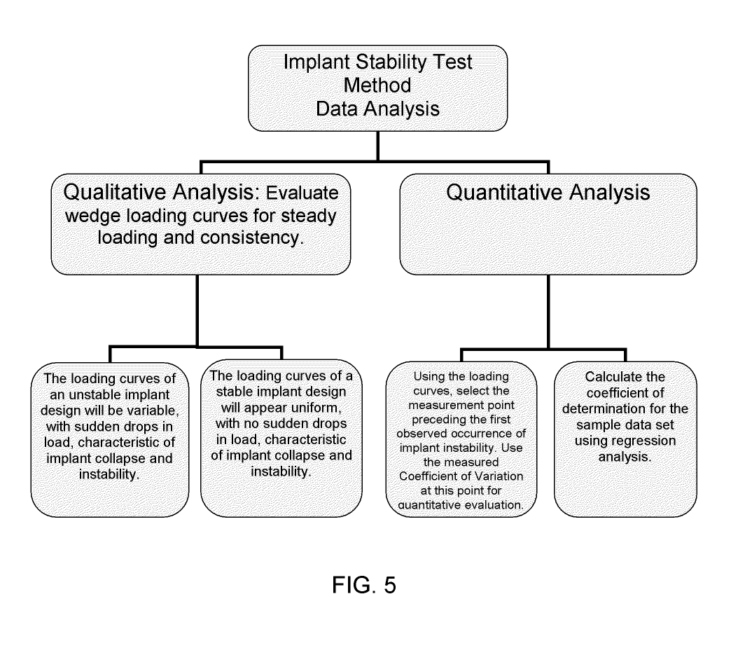

As depicted in the flowchart of FIG. 5, the stability of an implant can be evaluated qualitatively and/or quantitatively. For example, with respect to the qualitative analysis, the loading curves of a tested implant can be reviewed to ensure that there are no undesirable changes in load (e.g., drops in load as the implant is being extended) and/or to ensure a desired repeatability or uniformity of the load curves between sequential test runs of the same implant. As discussed in greater detail above, a relatively stable implant with not be subject to decreases (e.g., sudden drops) and/or other abrupt changes in load or force during the execution of a test procedure and will have generally repeatable or predictable load curves from one test run to the other. In contrast, in some embodiments, a relatively unstable implant can include decreases in load or force as the force applicator is moved toward the implant during the course of a test procedure. In addition, the load curves for relatively unstable implants will not be uniform, reproducible or otherwise predictable.

With continued reference to the flowchart of FIG. 5, an implant can be assigned a quantitative value of stability based on the data obtained using the test embodiments disclosed herein. For example, in some embodiments, the coefficient of variation can be calculated for the different test runs of the same implant at a point preceding implant canting, overturning or otherwise changing its longitudinal axis relative to the lumen within which it is positioned. In some embodiments, the point of canting or overturning is characterized by a decrease (e.g., sudden drop, other rapid change, etc.) in lateral or longitudinal force as the force applicator of the testing device is moved toward the implant. By way of example, for the implant being tested in relation to FIG. 4A, the coefficient of variation can be calculated at about 2.5 mm of extension of the force applicator, since it is at this extension distance that the implant canted or overturned (e.g., evidence by the drop in load) in one of the test runs. In some embodiments, the relative stability of two or more implants can be determined by comparing the coefficient of variation of such implants. However, in other embodiments, as discussed herein, in order to satisfy a required or desired level of lateral stability, the implant is compared to a target average coefficient of variation. For example, in some embodiments, in order to satisfy a lateral stability test, the tested implant can have an average coefficient of variation at or below about 20 (e.g., 10-12, 12-14, 14-16, 16-18, 18-20, values between the foregoing ranges, etc.). In other embodiments, the implant can have an average coefficient of variation below about 10 (e.g., 0-1, 1-2, 2-3, 3-4, 4-5, 5-6, 6-7, 7-8, 8-9, 9-10, values between the foregoing ranges, etc.). In other embodiments, the average coefficient of variation can be maintained below 50, 40, 30, 25 and/or other value above 20 in order for an implant to meet a threshold level of lateral stability.

In some embodiments, it may be desirable to require an implant to not fail (e.g., cant, overturn, otherwise move so as to cause a load drop during a test, etc.) before a particular load value in order for that implant to meet a threshold stability requirement. For example, in some embodiments, the implant must not cant or overturn before a lateral or longitudinal force of 5 N is applied to the implant by the force applicator. In some embodiments, the implant must withstand a minimum force of between 5 N and 10 N (e.g., 5 N, 5.5 N, 6 N, 6.5 N, 7 N, 7.5 N, 8 N, 8.5 N, 9 N, 9.5 N, loads between the foregoing values, etc.) in order to pass a stability threshold.

According to some embodiments, the coefficient of determination for the sample data set can be calculated using regression analysis. As discussed herein, in some embodiments, a coefficient of determination of the sample data set consisting of a series of test runs of the same implant greater than 0.7 (e.g., 0.7-0.75, 0.75-0.8, 0.8-0.85, 0.85-0.9, greater than 0.9, values between the foregoing ranges, etc.) can provide assurance that the implant has satisfied a desired or target stability threshold. In other embodiments, the target coefficient of determination is above 0.4 (e.g., 0.4-0.45, 0.45-0.5, values within the foregoing ranges, etc.), above 0.5 (e.g., (e.g., 0.5-0.55, 0.55-0.6, values within the foregoing ranges, etc.), above 0.6 (e.g., 0.6-0.65, 0.65-0.7, values within the foregoing ranges, etc.) and/or the like.

In some embodiments, if it is determined, following a test procedure, that an implant has not satisfied a threshold level of stability (e.g., the implant has an average coefficient of variation that is greater than a maximum target threshold, the implant has a coefficient of determined that is below a minimum target threshold, the implant cants or overturns before a minimum degree of movements of the test device's force applicator relative to the implant, etc.), one or more design features of the implant can be modified to improve the implant's lateral stability characteristics. For example, in some embodiments, the aspect ratio of the implant can be increased (e.g., by a particular incremental amount), the width of the ribbon of the implant can be increased, the length of the implant can be increased, the number of windings of the implant can be increased and/or the like. The aspect ratio is the ratio of implant length to diameter. Thus, for a given diameter and overall shape, the longer the implant, the more stable it is expected to be within the subject's body lumen. Further, in some embodiments, an implant having a higher aspect ratio may be selected when the width of the ribbon is reduced in order to maintain a desired level of lateral stability post- implantation. In some embodiments, after one or more design changes have been made to the implant, the implant can be retested using the embodiments disclosed herein to determine if the redesigned implant satisfies one or more target thresholds of stability (e.g., qualitative, quantitative, etc.).

To assist in the description of the disclosed embodiments, words such as upward, upper, bottom, downward, lower, rear, front, vertical, horizontal, upstream, downstream have been used above to describe different embodiments and/or the accompanying figures. It will be appreciated, however, that the different embodiments, whether illustrated or not, can be located and oriented in a variety of desired positions.

Although several embodiments and examples are disclosed herein, the present application extends beyond the specifically disclosed embodiments to other alternative embodiments and/or uses of the inventions and modifications and equivalents thereof It is also contemplated that various combinations or subcombinations of the specific features and aspects of the embodiments may be made and still fall within the scope of the inventions. Accordingly, it should be understood that various features and aspects of the disclosed embodiments can be combine with or substituted for one another in order to form varying modes of the disclosed inventions. Thus, it is intended that the scope of the present inventions herein disclosed should not be limited by the particular disclosed embodiments described above, but should be determined only by a fair reading of the claims that follow.

Various embodiments of the invention have been presented in a range format. It should be understood that the description in range format is merely for convenience and brevity and should not be construed as an inflexible limitation on the scope of the invention. The ranges disclosed herein encompass any and all overlap, sub-ranges, and combinations thereof, as well as individual numerical values within that range. For example, description of a range such as from 70 to 115 degrees should be considered to have specifically disclosed subranges such as from 70 to 80 degrees, from 70 to 100 degrees, from 70 to 110 degrees, from 80 to 100 degrees etc., as well as individual numbers within that range, for example, 70, 80, 90, 95, 100, 70.5, 90.5 and any whole and partial increments therebetween. Language such as “up to,” “at least,” “greater than,” “less than,” “between,” and the like includes the number recited. Numbers proceeded by a term such as “about” or “approximately” include the recited numbers. For example, “about 4 mm” includes “4 mm”.

While the inventions disclosed herein are susceptible to various modifications, and alternative forms, specific examples thereof have been shown in the drawings and are herein described in detail. It should be understood, however, that the inventions are not to be limited to the particular forms or methods disclosed, but to the contrary, the inventions are to cover all modifications, equivalents, and alternatives falling within the spirit and scope of the various embodiments described and the appended claims. Any methods disclosed herein need not be performed in the order recited. The methods disclosed herein include certain actions taken by a practitioner; however, they can also include any third-party instruction of those actions, either expressly or by implication. For example, actions such as “moving a force applicator” include “instructing the movement of a force applicator.”

What is claimed is:

1. A method of testing the stability of an implant configured for placement within a target vessel or other body lumen of a subject, comprising:
positioning an implant within a test conduit, the test conduit comprising a wall defining an interior opening, wherein the implant at least partially contacts an interior surface of the wall when positioned within the interior opening of the test conduit;
applying a longitudinal force to at least a portion of the implant using a force applicator configured to be selectively moved toward the implant and to engage the test conduit; and
evaluating a resistive force by the implant on the test conduit over a range of increasing longitudinal force applied to the implant by the force applicator;
wherein evaluating a resistive force by the implant on the test conduit over a range of increasing longitudinal force applied to the implant helps determine whether the implant maintains its longitudinal orientation within the test conduit.

2. The method of claim 1, wherein the force applicator comprises a curved or rounded shape along a location where the force applicator contacts the test conduit.

3. The method of claim 1, wherein the force applied to the at least a portion of the implant comprises both a radial force component and a longitudinal force component.

4. The method of claim 1, wherein evaluating a resistive force by the implant on the test conduit over a range of increasing longitudinal force applied to the implant comprises plotting the longitudinal force applied to the implant against a parameter associated with lateral stability of the implant in a graph.

5. The method of claim 1, wherein evaluating a resistive force by the implant on the test conduit over a range of increasing longitudinal force applied to the implant comprises determining whether a resistive force by the implant on the test conduit in the radial direction decreases as the longitudinal force applied to the implant increases.

6. The method of claim 1, wherein the implant comprises a ribbon that includes, at least in part, a helical or wound shape.

7. The method of claim 1, wherein the implant comprises a stent or any other intraluminal device configured to at least partially contact an adjacent portion of a body lumen when implanted in a subject.

8. The method of claim 1, wherein applying a force to at least a portion of the implant comprises applying a force along or near one end of the implant.

9. The method of claim 1, wherein a stability being evaluated comprises, at least in part, a lateral stability of the implant.

10. The method of claim 1, further comprising adjusting at least one parameter of the implant being tested to improve the stability of the implant.

11. The method of claim 10, wherein adjusting at least one parameter of the implant comprises at least one of increasing the aspect ratio of the implant, increasing a width of a winding or element of the implant, increasing a length of the implant.

12. A device for testing a lateral stability of an implant, comprising:
a test conduit comprising a wall defining an interior opening configured to receive an implant, wherein the implant at least partially contacts an interior surface of the wall when positioned within the interior opening of the test conduit; and
a force applicator configured to be selectively moved relative to the test conduit and contact at least a portion of the test conduit and configured to apply a force to at least a portion of the implant;
wherein the force application is configured to impart a lateral force component to the implant; and
wherein a parameter associated with the lateral stability of the implant within the test conduit is configured to be evaluated at various increasing levels of force applied to the implant.

13. The device of claim 12, wherein the force applicator comprises a curved or rounded shape along a location where the force applicator contacts the test conduit.

14. The device of claim 12, wherein the force application is configured to be moved using at least one of a pneumatic, hydraulic and mechanical device.

15. The device of claim 12, further comprising a platform configured to receive the test conduit during a testing procedure.

16. The device of claim 15, wherein the parameter associated with the lateral stability of the implant within the test conduit comprises a resistive force by the implant radially on an interior surface of the test conduit.

17. A device for testing a stability of an implant, comprising:
a test conduit comprising a wall defining an interior opening configured to receive an implant, wherein the implant at least partially contacts an interior surface of the wall when positioned within the interior opening of the test conduit; and
a force applicator configured to be selectively moved relative to the test conduit and contact at least a portion of the test conduit and configured to apply a force to at least a portion of the implant;
wherein a parameter associated with the lateral stability of the implant within the test conduit is configured to be evaluated at various increasing levels of force applied to the implant.

18. The device of claim 17, wherein the force application is configured to impart a lateral force component to the implant

19. The device of claim 17, wherein the force applied to the at least a portion of the implant comprises both a radial force component and a longitudinal force component.

20. The device of claim 17, wherein applying a force to at least a portion of the implant comprises applying a force along or near one end of the implant.
